(19) United States
(12) Patent Application Publication
(10) Pub. No.: US 2022/0120682 A1
(43) Pub. Date: Apr. 21, 2022

(54) METHOD FOR CHARACTERISING TARGET COMPOUNDS

(71) Applicant: ARYBALLE TECHNOLOGIES, Grenoble (FR)

(72) Inventors: Yanis Caritu, Grenoble (FR); Kirill Arkhipov, Grenoble Cecex 09 (FR)

(21) Appl. No.: 17/419,463

(22) PCT Filed: Dec. 28, 2019
(86) PCT No.: PCT/FR2019/053312

§ 371 Date: Jun. 29, 2021

(30) Foreign Application Priority Data
Dec. 31, 2018 (FR) 1874420

(51) Int. Cl. G01N 21/552; G01N 21/77

(52) U.S. Cl. CPC G01N 21/553; G01N 21/77

(57) ABSTRACT

Disclosed is a method for characterizing target compounds using an analyzing system comprising a measurement chamber intended to receive the target compounds contained in a fluid sample and in which a plurality of separate sensitive sites each comprise receivers able to interact with the target compounds. The method includes supplying a fluid sample, determining a measurement signal Sₖ(tᵢ) representative of the interactions between the target compounds and the receivers; computing a normed vector Sn(tᵢ); and reiterating the determining and computing steps, while incrementing the measurement time, until a stability criterion is met, so as to obtain a characterization of the target compounds from the normed vector Sn(tᵢ).

### CROSS-REFERENCE TO RELATED APPLICATIONS

This application is a national phase entry under 35 U.S.C. § 371 of International Patent Application PCT/FR2019/053312, filed Dec. 28, 2019, designating the United States of America and published as International Patent Publication WO 2020/141281 A 1 on Jul. 9, 2020, which claims the benefit under Article 8 of the Patent Cooperation Treaty to French Patent Application Serial No. 1874420, filed Dec. 31, 2018.

### TECHNICAL FIELD

The field of the disclosure is that of characterizing target compounds present in a fluid sample, preferably with an electronic nose employing a technology based on surface-plasmon-resonance imaging.

### BACKGROUND

The ability to characterize and to analyze target compounds, odor molecules or volatile organic compounds for example, contained in fluid samples is an increasingly important issue in various fields, and notably in those of health, and of fragrances in the perfume industry, in the food-processing industry, and with regard to olfactory comfort in confined public or private places (motor vehicle, hotel industry, shared places, etc.), etc.

Various characterization approaches exist, which differ from one another notably in that the target compounds or receptors need or do not need to be “labelled” beforehand with a marker. Unlike, for example, detection by fluorescence, which requires such markers to be used, detection by surface plasmon resonance (SPR) is a so-called label-free technique.

It may be implemented in an electronic nose, notably via SPR imaging, when the target compounds are contained in a gaseous or liquid sample. The surface-plasmon-resonance characterization technique allows an optical signal representative of adsorption and desorption interactions between target compounds and receptors placed on sensitive sites of the electronic nose to be measured in real time. Insofar as the chemical or physical affinity of interaction of the target compounds with the receptors is not known a priori, the characterization of the target compounds then consists in obtaining an interaction pattern from the optical signals measured for the sensitive sites. Adsorption on and desorption from a prepared (or “functionalized”) surface benefiting from differentiated adsorption characteristics allows the molecules present in the gas that have attached to the surface to be determined. The interaction of incident photons with the changing electronic cloud of the surface creates an energy transfer and updates the patterns according to the gaseous excitation.

In this regard, FIGS. 1A and 1B illustrate an example of an electronic nose such as described in patent application WO2018/158458. The electronic nose 1 generally comprises a fluid-supplying device 2, an optical device 3 for measuring by SPR imaging, and a processing unit (not shown).

The optical measuring device 3 comprises a measurement chamber 4 intended to receive the gaseous sample, in which chamber is located a measurement carrier 5 on which is located a matrix array of sensitive sites 6. The measurement carrier 5 is formed from a metal layer to which are fastened various receptors suitable for interacting with the target compounds, the various receptors being placed so as to form sensitive sites 6 that are distinct from one another. These receptors are then located at the interface between the metal layer and a dielectric medium, here a gaseous medium.

It further comprises a light source 7 for emitting exciting radiation, and an image sensor 8. The light source 7 is suitable for emitting exciting light radiation in the direction of the measurement carrier 5, at a working angle θR allowing surface plasmons to be generated thereon. The reflected portion of the exciting light radiation is then detected by the image sensor 8. The optical intensity of the reflected radiation depends locally on the refractive index of the measurement carrier 5, which itself depends on the surface plasmons generated and on the amount of material located at each sensitive site 6, this amount of material varying over time depending on the interactions between the sensitive compounds and the receptors.

The processing unit of the electronic nose is suitable for analyzing “sensorgrams,” i.e., the variation as a function of time in the optical intensity of the radiation reflected and measured by the image sensor 8, for each sensitive site 6, with the aim of extracting therefrom kinetic information on the interaction (adsorption and desorption) of target compounds with the receptors of the sensitive sites 6.

Finally, the fluid-supplying device 2 is suitable for introducing the target compounds into the measurement chamber 4, under conditions that allow analysis of the sensorgrams and therefore characterization of the target compounds.

In this regard, the article by Brenet et al. titled Highly-Selective Optoelectronic Nose based on Surface Plasmon Resonance Imaging for Sensing Gas Phase Volatile Organic Compounds, Anal. Chem. 2018, 90, 16, 9879-9887, describes a method for characterizing a gaseous sample by means of an electronic nose based on SPR imaging.

The characterizing method consists in supplying the measurement chamber with a gaseous sample in such a way that the kinetics of interaction between the target compounds and the receptors ensures a steady equilibrium state is reached.

More precisely, the fluid-supplying step comprises a first phase, called the initial phase, in which the gas sample is formed from the carrier gas alone, without target compounds; a second phase, called the injecting phase, in which the gas sample is formed from the carrier gas and the target compounds; and a third phase, called the dissociating phase, in which the target compounds are evacuated from the measurement chamber. The initial phase allows a reference optical signal intended to subsequently be subtracted from the measurement optical signal acquired in the injecting phase to be acquired. The injecting phase is carried out, via the fluid-supplying device, such that the sensorgrams reveal the presence of a transient assimilation state followed by a steady equilibrium state. When this steady equilibrium state is reached, characterization of the target compounds by the processing unit is then possible.

There is, however, a need to provide a characterizing method that would allow target compounds to be characterized in a simpler and faster manner, whether the steady adsorption/desorption equilibrium state has been reached between the target compounds and receptors or not.

### BRIEF SUMMARY

The objective of the disclosure is to at least partially remedy the drawbacks of the prior art, and more particularly to provide a method for characterizing target compounds that is simpler and faster, there being no need for the operating conditions as regards fluid supply and the structure of the fluid-supplying device to be engineered to obtain the steady adsorption/desorption equilibrium state within the measurement chamber. The characterizing method may be implemented using an electronic nose based on SPR technology, and preferably on SPR imaging, but also using other technologies, such as, for example, that of electromechanical resonators of the NEMS or MEMS type.

To this end, one subject of the disclosure is a method for characterizing target compounds, with an analyzing system comprising a measurement chamber intended to receive target compounds contained in a fluid sample, in which measurement chamber are located a plurality of distinct sensitive sites each comprising receptors that are able to interact with the target compounds, the method comprising the following steps:
fluidically supplying a fluid sample to the measurement chamber, this comprising an injecting phase P₂ in which the fluid sample is formed from a carrier fluid and the target compounds; and
determining, in the supplying step, at a measurement time tᵢ, for each sensitive site, a measurement signal Sₖ(tᵢ) representative of the interactions between the target compounds and the receptors, k being the rank of the sensitive site in question, so as to obtain a vector S(tᵢ), called the measurement vector, formed from the measurement signals Sₖ(tᵢ) acquired at the measurement time tᵢ.

According to the disclosure, the method furthermore comprises the following steps:
computing, at the measurement time tᵢ, a normalized vector Sn(tᵢ) from the measurement vector S(tᵢ) at the measurement time tᵢ, and from a norm ∥S(tᵢ)∥ computed from the measurement vector S(tᵢ) at the measurement time tᵢ; and
reiterating the steps of determining measurement signals and of computing the normalized vector, while incrementing the measurement time, until a stability criterion is met, so as to obtain a characterization of the target compounds on the basis of the normalized vector Sn(tᵢ) at the measurement time tᵢ.

The following are some preferred but non-limiting aspects of this characterizing method.

The fluid-supplying step may comprise, prior to the injecting phase P2, an initial phase P1 in which the fluid sample is formed from the carrier fluid without the target compounds. The step of determining the measurement signal Sₖ(tᵢ) may comprise computing a vector Su(tᵢ), called the useful vector, at the measurement time tᵢ, by subtracting from the measurement vector S(tᵢ) a vector S(Δt_{ref}), called the reference vector, determined in the initial phase P1 in a predetermined measurement period Δt_{ref}. The normalized vector Sn(tᵢ) may be computed from the useful vector Su(tᵢ).

The step of determining the measurement signal Sk(tᵢ) may comprise computing a vector Sc(tᵢ), called the corrected vector, from the measurement vector S(tᵢ) with application of a low-pass filter or from a sum of the values of the measurement vector S(tᵢ) at the previous measurement times.

The stability criterion may comprise a comparison, at the measurement time t₁, of a parameter Pₛₜ(tᵢ), called the stability parameter, computed from the coordinates Snₖ(tᵢ) of the normalized vector Sn(tᵢ) in a moving window tᵢ-Tₛₜ, to a determined threshold value P_{st,th}.

The stability parameter Pₛₜ(tᵢ) may be the maximum among the variances computed at the measurement time ti for the coordinates Snₖ(tᵢ-Tₛₜ) of the normalized vector Sn in a moving window tᵢ-Tₛₜ.

The stability criterion may comprise a comparison, at the measurement time tᵢ, of a parameter Pᵢₙⱼ(tᵢ), called the injection parameter, computed from the coordinates Sₖ(tᵢ) of the measurement vector S(tᵢ) in a moving window tᵢ-Tᵢₙⱼ, to a determined threshold value P_{inj,th}.

The injection parameter Pᵢₙⱼ(tᵢ) may be the maximum among the variances computed at the measurement time tᵢ for coordinates Sₖ(tᵢ-Tᵢₙⱼ) of the measurement vector S in a moving window tᵢ-Tᵢₙⱼ.

The norm ∥S(ti)∥ is preferably the Euclidean norm.

The characterizing step may comprise providing at least one parameter characteristic of a variation as a function of time in the Euclidean norm of the normalized vector Sn in the injecting phase P2.

The characterizing step may comprise computing an integral, over the duration of the injecting phase P2, of the Euclidean norm of the normalized vector Sn.

The analyzing system may be an electronic nose based on surface-plasmon-resonance imaging, or may be an analyzing system comprising a plurality of distinct electromechanical resonators each forming one sensitive site.

### BRIEF DESCRIPTION OF THE DRAWINGS

Other aspects, aims, advantages and features of the disclosure will become more clearly apparent on reading the following detailed description of preferred embodiments thereof, this description being given by way of nonlimiting example and with reference to the accompanying drawings, in which:

FIGS. 1A and 1B, which have already been described, are schematic and partial views, in cross section (FIG. 1A) and seen from above (FIG. 1B), of an electronic nose according to one example of the prior art and of the sensitive sites of a measurement carrier;

FIGS. 2A and 2B are examples of sensorgrams Suₖ(tᵢ) measured by the electronic nose, i.e., examples of the variation as a function of time in the optical intensity of the light radiation reflected by various sensitive sites and measured by the image sensor of the electronic nose, in the case of profiles that are said to be conventional (FIG. 2A) and in the case profiles that are said to be degraded (FIG. 2B);

FIG. 3 is a flowchart illustrating the various steps of a characterizing method according to one embodiment;

FIG. 4A illustrates the variation as a function of time in degraded-profile sensorgrams Suₖ(tᵢ), and the variation as a function of time (continuous bold line) in the injection parameter Pᵢₙⱼ(tᵢ) and the variation as a function of time (bold dashed line) in the threshold value P_{inj,th}(tᵢ) thereof, and FIG. 4B illustrates a partial and detailed view of FIG. 4A, showing more precisely the variations as a function of time in Pᵢₙⱼ(tᵢ) and P_{inj,th}(tᵢ), and allowing the phase P2 of injecting the target compounds in the fluid-supplying step to be identified;

FIGS. 5A and 5B illustrate one example of the variation as a function of time (FIG. 5A) in corrected signals Scₖ(tᵢ) obtained by summing degraded-profile sensorgrams Suₖ(tᵢ) that are identical or similar to those illustrated in FIG. 2B, and one example of the variation as a function of time (FIG. 5B) in normalized signals Snₖ(tᵢ) and the variation as a function of time in the corresponding stability parameter Pₛ(tᵢ);

FIGS. 6A and 6B illustrate one example of the variation as a function of time (FIG. 6A) in corrected signals Scₖ(tᵢ) obtained by applying low-pass filtering to degraded-profile sensorgrams Suₖ(tᵢ) that are identical or similar to those illustrated in FIG. 2B, and one example of the variation as a function of time (FIG. 6B) in normalized signals Snₖ(tᵢ) and the variation as a function of time in the corresponding stability parameter Pₛ(tᵢ); and

FIG. 7A illustrates one example of an interaction pattern obtained with the characterizing method according to one embodiment, in the case where the sensorgrams have a degraded profile, and FIG. 7B illustrates one example of the variation as a function of time in an instantaneous interaction intensity Iᵢₙₜ(tᵢ) associated with sensorgrams that are identical or similar to those illustrated in FIG. 2B.

### DETAILED DESCRIPTION

In the figures and in the remainder of the description, the same references have been used to designate identical or similar elements. In addition, the various elements have not been shown to scale so as to improve the clarity of the figures. Moreover, the various embodiments and variants are not mutually exclusive and may be combined with one another. Unless otherwise indicated, the terms “substantially,” “about” and “of the order of” mean to within 10%, and preferably to within 5%.

The disclosure relates to characterizing target compounds contained in a fluid sample by means of an analyzing system comprising a measuring device, a fluid-supplying device and a processing unit. As detailed below, the measuring device comprises a measurement chamber that is suitable for receiving a fluid (gaseous or liquid) sample comprising the target compounds, in which measurement chamber are located a plurality of distinct sensitive sites, each comprising at least one receptor suitable for interacting, by adsorption/desorption, with the target compounds.

In the remainder of the description, the analyzing system is an electronic nose based on surface-plasmon-resonance (SPR) imaging. However, other characterization technologies may be used. In this regard, the analyzing system may, as an alternative to the electronic nose, comprise MEMS resonators (MEMS being the acronym of micro-electro-mechanical system) or NEMS resonators (NEMS being the acronym of nano-electro-mechanical system). This type of technology is known to those skilled in the art, and an example of such an analyzing system is described in document EP3184485. The analyzing system comprises a plurality of electromechanical resonators that are distinct from one another, a surface of each resonator being functionalized by the presence of receptors, and thus forming a sensitive site. In a known manner, the interactions between the target compounds and the receptors of a sensitive site cause a modification of the resonant frequency of the electromechanical resonator. In a similar way to the SPR technology of the electronic nose, measuring the variation in the resonant frequency thus allows a measurement signal Sₖ(tᵢ) representative of the interactions between the target compounds and the receptors of the sensitive site of rank k in question, to be obtained. The variation in the resonant frequency may be measured via a piezoresistive or capacitive measurement, inter alia.

Generally, by characterization, what is meant is obtaining information representative of the interactions of the target compounds contained in the fluid sample with the receptors of various sensitive sites of the analyzing system. The interactions in question are here events resulting in the target compounds adsorbing on and/or desorbing from the receptors. This information thus forms an interaction pattern, or in other words a “signature” of the target compounds, this pattern being representable, for example, in the form of a histogram or of a radar chart. More precisely, in the case where the analyzing system comprises N distinct sensitive sites, the interaction pattern is formed by N representative items of information, these being formed of a value correlated to the intensity of a measurement optical signal obtained for the sensitive site in question.

Generally, the target compounds are elements intended to be characterized by the electronic nose, and contained in a fluid sample. They may be, by way of illustration, bacteria, viruses, proteins, lipids, volatile organic molecules, inorganic compounds, inter alia. Moreover, the receptors are elements that are fastened to the sensitive sites and that exhibit a capacity for interaction with the target compounds, though the chemical and/or physical affinities between the sensitive compounds and the receptors are not necessarily known. The receptors of the various sensitive sites preferably have different physico-chemical properties, which have an impact on their ability to interact with the target compounds. It may be a question, by way of example, of amino acids, peptides, nucleotides, polypeptides, proteins, organic polymers, inter alia.

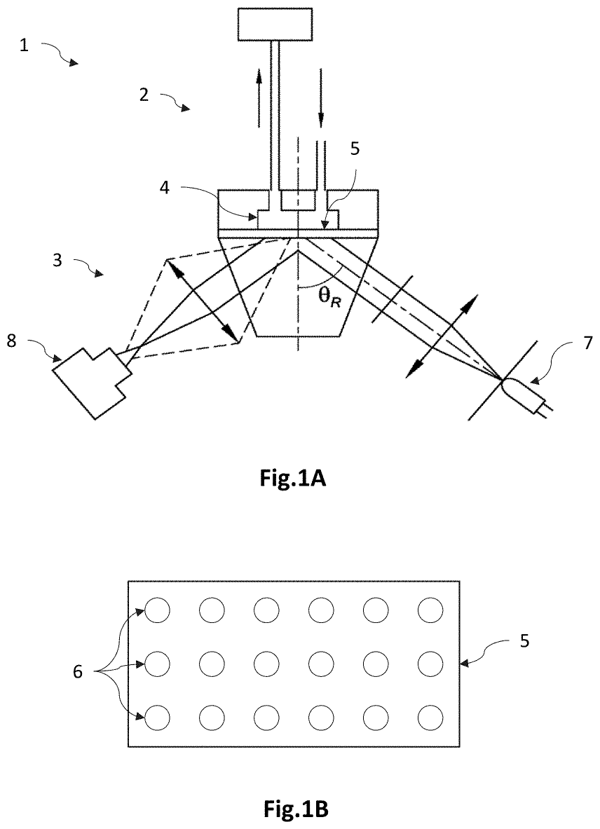

With reference to FIGS. 1A and 1B, which were briefly described above, the electronic nose 1 is an optoelectronic system allowing target compounds, for example, odor molecules or volatile organic compounds inter alia, contained in a fluid sample introduced into a measurement chamber 4 of the electronic nose, to be characterized. The electronic nose 1 shown in these figures here has the features of the so-called Kretschmann configuration, which is known to those skilled in the art, though the disclosure is not however limited to this configuration. The fluid sample may be a liquid or a gaseous sample. In the remainder of the description, it is a question of a gaseous sample.

The electronic nose 1 comprises, located in a measurement chamber 4 intended to receive the gaseous sample to be analyzed, a plurality of sensitive sites 6 that are distinct from one another, each formed from receptors that are able to interact with the target compounds to be studied and therefore able to interact in a differentiated manner with the sample. The sensitive sites 6 are distinct from one another in the sense that they comprise receptors that are different, in terms of chemical or physical affinity with respect to the target compounds to be analyzed, and are therefore intended to deliver interaction information that differs from one sensitive site 6 to the next. The sensitive sites 6 are distinct regions of a measurement carrier 5, and may be contiguous or spaced apart from one another. The electronic nose 1 may further comprise a plurality of identical sensitive sites 6, for example with the aim of detecting any measurement drift or of identifying a defective sensitive site.

The electronic nose comprises an optical measuring device 3 of SPR-imaging type, allowing, for each sensitive site 6, the interactions of the target compounds with the receptors to be quantified, here via measurement of the intensity of an optical signal associated with the sensitive site 6 in question, this optical signal being a portion, here a reflected portion, of the exciting light radiation emitted by a light source. The intensity of the measured optical signal is directly correlated with the interactions between the target compounds and the receptors.

In the context of measurement by SPR imaging, the optical measuring device 3 is suitable for acquiring, in real time, light radiation originating from all of the sensitive sites 6. Thus, the optical signals emitted by the plurality of sensitive sites 6 are measured together and in real time, in the form of an image acquired by the same optical sensor 8.

Thus, the optical measuring device 3 comprises a light source 7 suitable for transmitting so-called exciting light radiation in the direction of sensitive sites 6, and for generating surface plasmons on the measurement carrier 5. The light source 7 may be formed from a light-emitting diode, the emission spectrum of which has an emission peak centered on a central wavelength λ_{c}. Various optical elements (lenses, polarizer, etc.) may be placed between the light source 7 and the measurement carrier 5.

The optical measuring device 3 further comprises an image sensor 8, i.e., a matrix-array optical sensor suitable for collecting an image of the light radiation that originates from the sensitive sites in response to the exciting light radiation. The image sensor 8 is a matrix-array photodetector, a CMOS or CCD sensor for example. It therefore comprises a matrix array of pixels the spatial resolution of which is such that, preferably, a plurality of pixels acquires a portion of the reflected light radiation associated with a given sensitive site 6.

The processing unit (not shown) allows the processing operations described below in the context of the characterizing method to be implemented. It may comprise at least one microprocessor and at least one memory. It is connected to the optical measuring device 3, and more precisely to the image sensor 8. It comprises a programmable processor able to execute instructions stored on a data storage medium. It further comprises at least one memory containing the instructions required to implement the characterizing method. The memory is also suitable for storing the information computed at each measurement time.

As described below, the processing unit is notably suitable for storing and processing a plurality of images, called elementary images, acquired at a given sampling frequency fₑ in a measurement period Δt, in order to determine, at the current time tᵢ, an optical measurement signal Sₖ(tᵢ) associated with the sensitive site of rank k.

The fluid-supplying device 2 is suitable for supplying the measurement chamber 4 with gaseous samples, formed from a carrier gas with or without target compounds. As mentioned above, the fluid samples may, as a variant, be in the liquid phase (carrier liquid, with or without target compounds). To this end, the device comprises a reservoir of carrier gas, and a source of target compounds. It may comprise a plurality of fluid lines, connected to the inlet of the measurement chamber 4, and may comprise valves and mass flow regulators. It thus allows the measurement chamber 4 to be supplied with at least one carrier gas without target compounds, for example in the initial phase and the dissociating phase, and allows the target compounds to be injected in the injecting phase. It may be able to ensure that the concentration of the target compounds in the measurement chamber remains constant over time, or not.

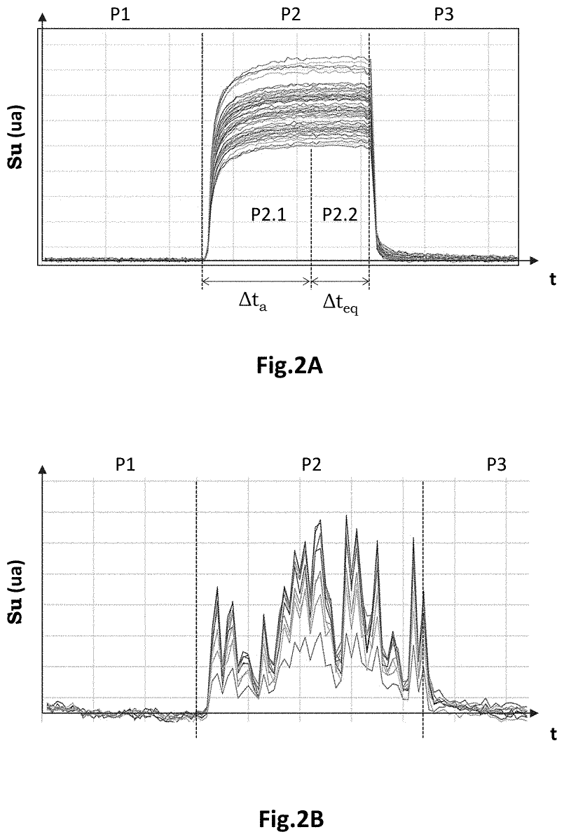

FIG. 2A illustrates an example of sensorgrams Suₖ(t), each being associated with one sensitive site of the electronic nose, in the context of a characterizing method in which the sensorgrams each have a profile that is said to be conventional, i.e., they reveal the presence of a steady equilibrium state between the target compounds and the receptors, as explained below.

A sensorgram corresponds to the variation as a function of time in a signal representative of the interactions between the target compounds and the receptors of a sensitive site. In this example, it is a question of the intensity of an optical signal Suₖ(t), called the useful signal, associated with the sensitive site of rank k, and more precisely here a question of the variation in reflectivity ΔR corresponding to the modification of the refractive index, of the sensitive site of rank k in question, related to the adsorption and desorption interactions of the target compounds with the receptors of the sensitive site.

In a known manner, a conventional-profile sensorgram exhibits an initial phase P1, a phase P2 of injecting target compounds, then a dissociating phase P3. The intensity of the sensorgram signal is proportional to the number of receptors of the sensitive site in question.

The initial phase P1 corresponds to the introduction into the measurement chamber of the carrier fluid alone, without target compounds. The sensorgrams thus represent a reference signal characteristic of the measurement environment. This reference signal is intended to be subsequently subtracted from the measurement signal to thus obtain a useful signal representative of the interactions of the target compounds.

The phase P2 of injecting target compounds corresponds to the introduction, into the measurement chamber, of a fluid sample formed from the carrier fluid and from the target compounds. This phase comprises a transient assimilation state P2.1 followed by a steady equilibrium state P2.2.

The transient assimilation state P2.1 corresponds to the gradual but exponential increase (Langmuir's law) in the interactions between the target compounds and the receptors, as the target compounds are injected into the measurement chamber. The exponential growth of the sensorgrams in the assimilation state is due to the fact that there are, then, many more adsorption events than desorption events.

It will be noted that, in this regard, the interaction between a target compound A (A standing for analyte) and a receptor L (L standing for ligand) is a reversible effect characterized by a constant kₐ (in mol⁻¹.s⁻¹) of adsorption of the target compound A on the receptor L to form a target compound/receptor LA (LA standing for ligand-analyte), and by a constant k_{b} (in s⁻¹) of desorption corresponding to the dissociation of the compound LA. The ratio k_{d}/kₐ forms the equilibrium dissociation constant k_{D} (in mol) that gives the value of the concentration c_{A} of target compounds A allowing 50% of the receptors L to be saturated.

The steady equilibrium state P2.2 is reached when the concentration c_{LA}(t) in compounds LA remains constant dc_{LA}/dt=0, i.e., when the product of the constant kₐ and the concentrations of target compounds c_{A}(t) and of receptors c_{L}(t) (number of adsorption events) is equal to the product of the constant k_{d} and the concentration c_{LA}(t) of compounds LA (number of desorption events), or in other words when the following rate equation is respected dc_{LA}/dt=kₐ×c_{A}×c_{L}−k_{d}×c_{LA}=0. The maximum steady-state value of the response signal is proportional to the concentration c_{A}(t) of target compounds A. Saturation of the receptors L at the sensitive site may be achieved when the concentration c_{A} of target compounds A is sufficient.

The dissociating phase P3 corresponds to a step of removing the target compounds present in the measurement chamber, so that the concentration of compounds LA decreases, usually exponentially.

It should be clear from the rate equation indicated above that the steady equilibrium state P2.2 requires the concentration c_{A}(t) of target compounds A in the measurement chamber to remain constant in the injecting step P2. The equilibrium state cannot therefore be reached when the concentration c_{A}(t) of target compounds A varies over time. This therefore requires the fluid-supplying device of the electronic nose to be able to precisely control the concentration c_{A}(t) of the target compounds in the measurement chamber, and the characterizing method to comprise a rigorous protocol for fluidically managing the target compounds, as well as strict and controlled operating conditions.

FIG. 2B illustrates another example of sensorgrams Suₖ(t) associated with the sensitive sites of the electronic nose. They differ from those illustrated in FIG. 2A notably in that the profiles do not exhibit a steady equilibrium state P2.2, and are thus said to be degraded.

The sensorgrams Suₖ(t) are obtained in a fluid-supplying step that also comprises an initial phase P1, a phase P2 of injecting the target compounds and a dissociating phase P3. The initial and dissociating phases P1, P3 are here similar to those described above. In contrast, in the injecting phase P2, it may be seen that the sensorgrams Suₖ(t) exhibit large intensity variations, and hence it is possible neither to identify any transient assimilation state P2.1 nor to identify a steady equilibrium state P2.2.

This type of degraded sensorgram profiles may be representative of a situation in which the concentration c_{A}(t) of target compounds within the measurement chamber varies over time. A steady state of equilibrium between the rates at which the target compounds adsorb on and desorb from the receptors cannot then be obtained. These sensorgrams may be obtained by means of a simplified fluid-supplying device that does not allow the value of the concentration c_{A}(t) over time to be controlled, and notably when the electronic nose is used under real, uncontrolled conditions, when the target compounds do not have a constant concentration c_{A}(t). By way of example, it may thus be a question of injection of an odor present in the open air and the concentration of which cannot be controlled.

The characterizing method according to the disclosure allows the target compounds to be characterized on the basis of sensorgrams having conventional or degraded profiles, i.e., whether the concentration c_{A}(t) of the target compounds in the measurement chamber is constant or not. This characterizing method further has the advantage of being able to provide additional information relative to the target compounds, such as an instantaneous interaction intensity Iᵢₙₜ(tᵢ) representative of the instantaneous population of target compounds adsorbed by the receptors, and a total level of exposure of the receptors to the target compounds in the period of the injecting phase P2. The “instantaneous” character is here relative to the integration time of the image sensor, its acquisition period Δt, etc., and not to the effective characteristic times of the (much faster) physical adsorption/desorption effects.

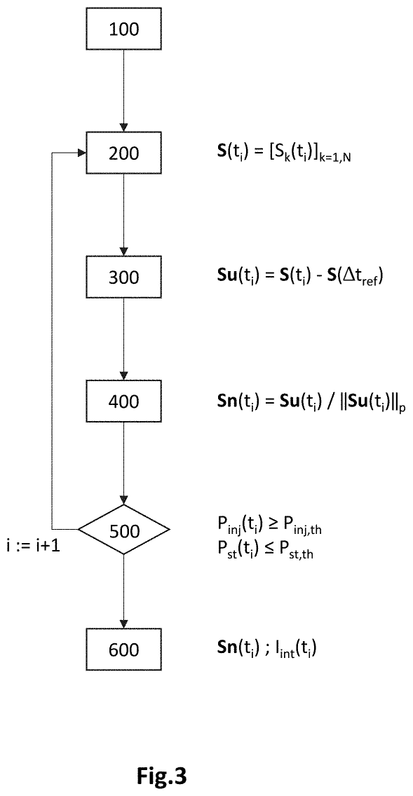

FIG. 3 illustrates a flowchart of a method for characterizing target compounds according to one embodiment. The fluid sample is here a gaseous sample.

In a first step 100, the fluid supply of the measurement chamber of the electronic nose with a gaseous sample is activated. This step comprises an initial first phase P1, a phase P2 of injecting target compounds, then a dissociating phase P3.

In this example, the fluid-supplying device is suitable for injecting target compounds into the measurement chamber without the concentration c_{A}(t) necessarily remaining constant. The concentration c_{A}(t) may remain constant, just as it may exhibit substantial variations as a function of time. In any case, the concentration c_{A}(t) is assumed to remain constantly above a minimum concentration corresponding to the detection limit of the electronic nose. Thus, the injecting phase does not necessarily exhibit a steady equilibrium state.

The following steps of determining the measurement signals and of processing the data are carried out in the fluid-supplying step, and reiterated for a plurality of successive measurement times tᵢ, until a stability criterion is met. With each iteration i is thus associated one measurement time tᵢ, also called the current time.

In a step 200, for each sensitive site of rank k ranging from 1 to N, at the current time tᵢ, a measurement signal Sₖ(tᵢ) representative of the interactions between the target compounds and the receptors is determined, at a measurement time tᵢ, in order thus to obtain a measurement vector S(tᵢ).

More precisely, the image sensor acquires, in a period Δt separating two successive measurement times tᵢ₋₁ and tᵢ, a plurality of images Ieₘ, called elementary images, of the matrix array of N distinct sensitive sites, m being the acquisition rank of the elementary image Ie, at a sampling frequency fₑ. The sampling frequency fₑ may be 10 images per second, and the acquisition period Δt may be a few seconds, 4 s for example.

For each elementary image Ieₘ, the processing unit determines an elementary optical intensity value (sₖ)ₘ by taking the average of the optical intensity (sₖ(i,j))ₘ acquired by each pixel i, j associated with a given sensitive site of rank k, and computes an average value (s̅ₖ̅)_{Δt} thereof over the acquisition period Δt. This average value (s̅ₖ̅)_{Δt} then corresponds to the optical measurement signal Sₖ(tᵢ), at the current time tᵢ, associated with the sensitive site of rank k.

A measurement vector S(tᵢ) is thus obtained, at the current time tᵢ, the coordinates [S₁(tᵢ), . . . , Sₖ(tᵢ), . . . S_{N}(tᵢ)] of which are the measurement signals of the sensitive sites at the current time tᵢ, and hence it is possible to write: S(tᵢ)=[Sₖ(tᵢ)]_{k=1,N}.

In a step 300, a useful vector Su(tᵢ) is advantageously computed sat the current time tᵢ, by subtracting from the measurement vector S(tᵢ) determined beforehand a reference value acquired for each sensitive site k in the initial phase P1, so that Su(tᵢ)=S(tᵢ)−S(Δt_{ref})=[Sₖ(tᵢ)]_{k=1,N}−[Sₖ(Δt_{ref})]_{k=1,N}. The reference period Δt_{ref} is, for example, equal to several times the acquisition period Δt in the initial phase P1, and may be a period that directly precedes the injecting phase P2. Thus, information associated with the carrier gas alone, given by S(Δt_{ref}), is subtracted from the information contained in S(tᵢ), allowing information related essentially to the interactions between the target compounds and the receptors to be revealed.

In a step 400, a normalized vector Sn(tᵢ) at the current time tᵢ is computed from the measurement vector S(tᵢ) at the current time tᵢ and from a norm of the measurement vector S(tᵢ) at the current time tᵢ. Preferably, the ratio between the useful vector Su(tᵢ) at the current time tᵢ and a norm ∥Su(tᵢ)∥ thereof at the current time tᵢ is computed.

It will be noted that, generally, the norm of order p of a vector S of size N and of coordinates [Sₖ]_{k=1,N} is defined by the following relationship: ∥S∥ₚ=(Σ_{k=1,N} |Sₖ|^{p})^{1/p}, with p a non-zero, positive integer or decimal number. Thus, the norm 1 corresponds to the sum of the absolute value of the coordinates of the vector S, and the norm 2 corresponds to the Euclidean norm.

In this example, the Euclidean norm (norm 2) is preferably used. Thus, the normalized vector Sn(tᵢ) at the current time tᵢ is computed via the following relationship:

$$Sn ( {t}_{i} ) = Su ( {t}_{i} ) / {( {\sum }_{k = 1 , N} {\vert {Su}_{k} ( {t}_{i} ) \vert}^{2} )}^{1 / 2} .$$

Preferably, the useful vector Su(tᵢ) at the current time tᵢ may have been processed beforehand to obtain a vector Sc(tᵢ), called the corrected vector, from which noise has been at least partially filtered and/or the signal-to-noise ratio of which has been increased. Thus, by way of example, the corrected vector Sc(tᵢ) may be obtained by applying a classical low-pass filter to the useful vector Su(tᵢ), or even, as a variant, by computing the sum of the preceding measurement times: Sc(tᵢ)=Su(tᵢ)+Sc(tᵢ₋₁).

As described below, it turns out, surprisingly, that sensorgrams formed on the basis of such a normalized vector Sn(tᵢ) exhibit, during the injecting phase P2, a transient portion of a particularly short duration, followed by a steady-state portion. It will be noted that these transient and steady-state portions do not correspond to the assimilation and equilibrium states described above, insofar as, in the injecting phase, equilibrium is not reached between the rates at which the target compounds adsorb on and desorb from the receptors. Nonetheless, it is however possible to characterize the target compounds on the basis of the steady-state portion of the sensorgrams Snₖ(tᵢ).

In a step 500, a stability criterion is computed that is such that, once it is met, the characterizing method may start to characterize the target compounds, i.e., to generate an interaction pattern, for example one taking the form of a histogram or a radar chart, from the coordinates of the normalized vector Sn(tᵢ) at the current time tᵢ. In this example, the stability criterion is met when the normalized vector Sn(tᵢ) exhibits a sufficient stability as a function of time in the injecting phase P2.

Thus, an advantageous first sub-step 510 may consist in identifying the injecting phase P2 in the fluid-supplying step. To this end, one approach is to determine a parameter Pᵢₙⱼ(tᵢ), called the injection parameter, at the current time tᵢ. This injection parameter Pᵢₙⱼ(tᵢ) may be defined as being equal to the maximum of the variance Vₖ, computed, at the current time tᵢ, in a moving window tᵢ-Tᵢₙⱼ, of the useful signals Suₖ(tᵢ) (or of the corrected signals Scₖ(tᵢ)). The period Tᵢₙⱼ of the moving window is equal to a plurality of times the acquisition period Δt, and for example is equal to 5×Δt. Thus, the injection parameter is written:

$${P}_{inj} ( {t}_{i} ) = {max}_{k = 1 , N} {V}_{k} ( {Su}_{k} ( {t}_{i} - {T}_{inj} ) ) .$$

Other formulations of the injection parameter are also possible (average of a time difference, etc.).

The injecting phase may be said to be identified when the injection parameter Pᵢₙⱼ(tᵢ) is higher than or equal to a threshold value P_{inj,th}: Pᵢₙⱼ(tᵢ)≥P_{inj,th}. This threshold value P_{inj,th} may be predetermined, for example from a pre-filled database relating to various target compounds, or may be determined during the characterizing method. In this regard, the threshold value P_{inj,th} may be set, in the initial phase P1 of the supplying step, equal to the product of a coefficient and of the maximum of the values of the injection parameter Pᵢₙⱼ(tᵢ) in a training period Tₗₑₐᵣₙ, for example equal to 10 or 20 times Δt, in the initial phase P1. The coefficient may be equal to 1.02, 1.05, 1.10, inter alia. Thus,

$${P}_{inj , th} = 1.05 \times {max}_{{T}_{learn}} {P}_{inj} ( {t}_{i} )$$

is computed. Next, after this training period Tₗₑₐᵣₙ, the threshold value P_{inj,th} is set and remains constant throughout the fluid-supplying step. Retraining may then be carried out when the injection parameter falls and persistently remains below the threshold. This allows a reference that is advantageously more recent to be obtained.

Next, a second sub-step 520 may consist in determining the temporal stability of the normalized vector Sn(tᵢ) in the fluid-supplying step. To this end, one approach is to determine a parameter Pₛₜ(tᵢ), called the stability parameter, at the current time tᵢ. This stability parameter Pₛₜ(tᵢ) may be defined as being equal to the maximum of the variance Vₖ, computed, at the current time tᵢ, in a moving window tᵢ-Tₛₜ, of the useful signals Snₖ(tᵢ). The period Tₛₜ of the moving window may be different from or equal to the period Tᵢₙⱼ, and may be equal to a plurality of times the acquisition period Δt, and for example is equal to 5×Δt. Thus, the stability parameter is written:

$${P}_{st} ( {t}_{i} ) = {max}_{k = 1 , N} {V}_{k} ( {Sn}_{k} ( {t}_{i} - {T}_{st} ) ) .$$

The temporal stability may be said to be sufficient when the stability parameter Pₛₜ(tᵢ) is lower than or equal to a threshold value P_{st,th}: Pₛₜ(tᵢ)≤P_{st,th}. This threshold value P_{st,th} may be predetermined, for example from a pre-filled database relating to various target compounds, or may be determined during the characterizing method. In this regard, the threshold value P_{st,th} may be set, in real time in the supplying step, equal to the product of a coefficient and of the maximum of the values of the stability parameter Pₛₜ(tᵢ) in the training period Tₗₑₐᵣₙ in the initial phase P1. The coefficient may be equal to 1.02, 1.05, 1.10, inter alia. Thus,

$${P}_{st , th} = 1.05 \times {max}_{{T}_{learn}} {P}_{st} ( {t}_{i} )$$

is computed. Next, after this training period Tₗₑₐᵣₙ, the threshold value P_{st,th} is set and remains constant throughout the fluid-supplying step.

In the case where a single condition is met or no conditions are met, the stability criterion is not met and the method reiterates steps 200 to 500 of acquiring images and processing data. Thus, in the case where Pᵢₙⱼ(tᵢ)<P_{inj,th}, the supplying step is considered to still be in the initial phase P1 of the fluid-supplying step, and the gas sample is therefore considered to contain only the carrier gas and not yet the target compounds. In the case where Pₛₜ(tᵢ)>P_{st,th}, the supplying step is considered to be in the injecting phase P2 of the fluid-supplying step, and the gas sample is therefore considered to contain the target compounds, but that the temporal stability of the normalized vector Sn(tᵢ) is considered to be insufficient to allow the target compounds to be characterized. In contrast, the stability criterion is said to be met when both the above conditions are met, namely when Pᵢₙⱼ(tᵢ)≥P_{inj,th}, and when Pₛₜ(tᵢ)≤P_{st,th}. The normalized vector Sn(tᵢ) is then considered to have a sufficient stability to allow the target compounds then present in the gas sample to be characterized, and the method passes to the next step of generating the interaction pattern.

In a step 600, the target compounds are characterized on the basis of the normalized vector Sn(tᵢ) at the current time tᵢ. It is then a question of generating a representation, taking the form of a histogram or radar chart, inter alia, of the coordinates Snₖ(tᵢ) of the normalized vector at the current time tᵢ, which corresponds to the final measurement time. Advantageously, the method for characterizing target compounds allows, in addition to the interaction pattern, additional information, such as the total number of interactions between the target compounds and the receptors in the injecting phase P2, and the variation as a function of time in an interaction intensity, to be generated. In this regard, the interaction intensity Iᵢₙₜ(tᵢ) at the current time tᵢ corresponds, for example, to the Euclidean norm (norm 2) of the normalized vector Sn(tᵢ), and the total number of interaction corresponds to the integral, over the injecting phase P2, of the intensity of interaction Iᵢₙₜ(tᵢ).

Thus, as detailed below, the characterizing method allows an interaction pattern describing the interaction of the target compounds with the receptors of the sensitive sites to be generated in a faster and simpler manner than in the case of the example of the prior art mentioned above. Specifically, it turns out that the normalized vector Sn(tᵢ) exhibits a steady-state portion in the phase P2 of injecting the target compounds. The interaction pattern may therefore be obtained on the basis of the normalized vector Sn(tᵢ) as soon as the stability criterion indicates that the steady-state portion has been reached. It would therefore appear, in this regard, that this steady-state portion is preceded by a very short transient portion, which reflects the start of the injecting phase P2. It is thus possible to generate the interaction pattern in a short time, much shorter than in the case of the prior art insofar as it is no longer necessary to wait for the steady equilibrium state P2.2 to be reached.

In addition, the characterizing method may generate the interaction pattern even if the concentration c_{A}(t) of target compounds in the measurement chamber is not constant, and therefore even if the sensorgrams do not exhibit the steady equilibrium state P2.2. It is thus possible to characterize the target compounds under “real conditions,” i.e., under simplified operating conditions and with a simplified fluid-supply protocol. This thus simplifies the characterizing method, and also decreases the structural complexity of the fluid-supplying device, notably in terms of valves and of mass flow controller. Simultaneously, this normalized indicator provides access to a window onto the competition that could occur in the inter-site kinetics and be a characteristic of the target compounds studied.

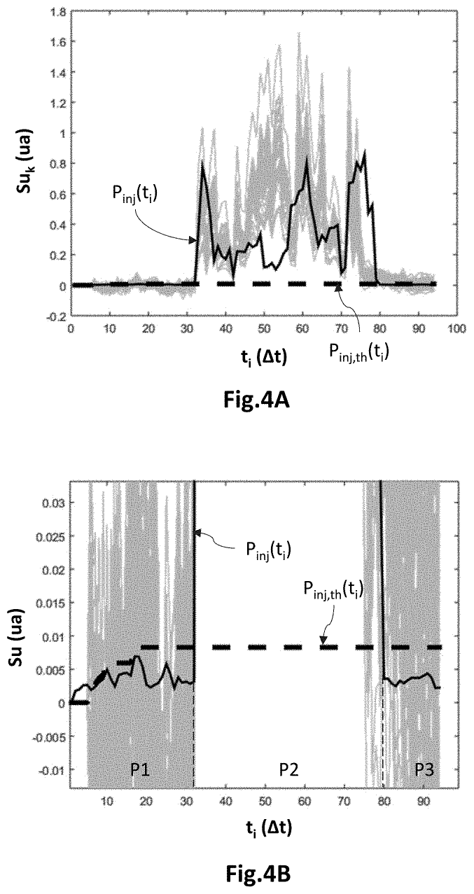

FIGS. 4A and 4B illustrate the sub-step 510 of identifying the injecting phase P2. FIG. 4A shows the variation as a function of time in the useful signals Suₖ(tᵢ), the variation as a function of time in the injection parameter Pᵢₙⱼ(tᵢ), and the variation as a function of time in its threshold value P_{inj,th} (tᵢ), in the case of degraded-profile sensorgrams similar to those in FIG. 2B. FIG. 4B shows part of FIG. 4A in detail. FIG. 4B, in particular, illustrates (dashed line) the variation as a function of time in the injection parameter Pᵢₙⱼ(tᵢ), and (continuous line) the variation as a function of time in the threshold value P_{inj,th}(tᵢ). It may be seen that the threshold value P_{inj,th}(tᵢ) is in a training phase in the initial phase P1 of the fluid-supplying step, and then has a set and constant value. The injection parameter Pᵢₙⱼ(tᵢ) allows, in the period Tₗₑₐᵣₙ, the set final value of P_{inj,th} to be defined. Subsequently, its variation as a function of time allows the injecting phase P2, then the dissociating phase P3, to be identified, depending on whether Pᵢₙⱼ(tᵢ) is higher than or equal to P_{inj,th} or lower.

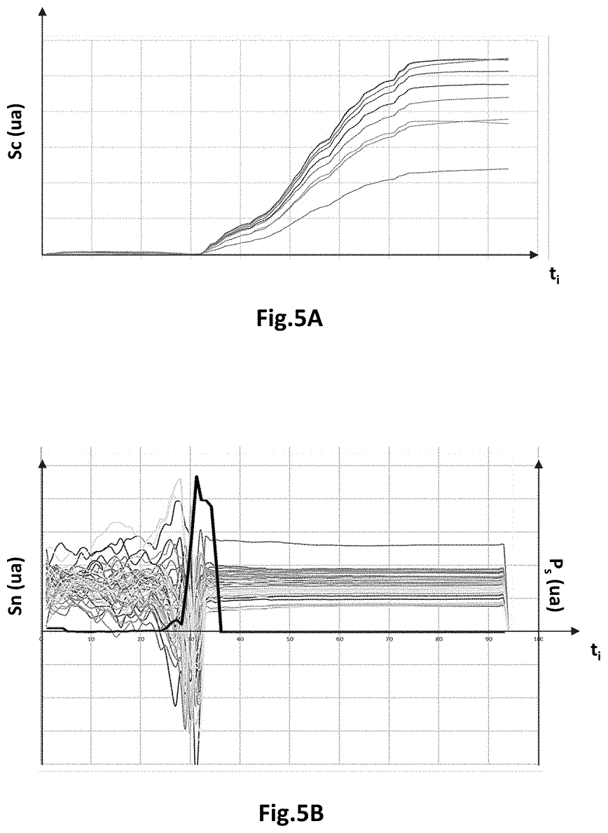

FIGS. 5A and 5B illustrate one example of the variation as a function of time (FIG. 5A) in the corrected vector Sc(tᵢ) computed from the useful vector Su(tᵢ), and one example of the variation as a function of time (FIG. 5B) in the normalized vector Sn(tᵢ) computed from the corrected vector Sc(tᵢ), and the variation as a function of time in the stability parameter Pₛₜ(tᵢ). These vectors were also obtained from degraded-profile sensorgrams. The corrected vector Sc(tᵢ) is computed from the sum of the values of the useful vector: Sc(tᵢ)=Su(tᵢ)+Sc(tᵢ). This approach allows a corrected vector having a higher signal-to-noise ratio to be obtained. The stability parameter Pₛₜ(tᵢ) exhibits a first portion with an almost zero value in the initial phase P1, followed by a transient portion with a high value at the start of the injecting phase P2, then a steady-state portion with an almost zero value in the injecting phase P2. This steady-state portion is then used to characterize the target compounds. It may be seen that the duration of the transient phase is particularly short, and much shorter than the duration of the transient assimilation regime P2.1 in the case of conventional-profile sensorgrams. It is thus possible to characterize the target compounds much more quickly than in the case of conventional characterizing methods, whether or not an equilibrium P2.2 is reached between the absorption and desorption rates.

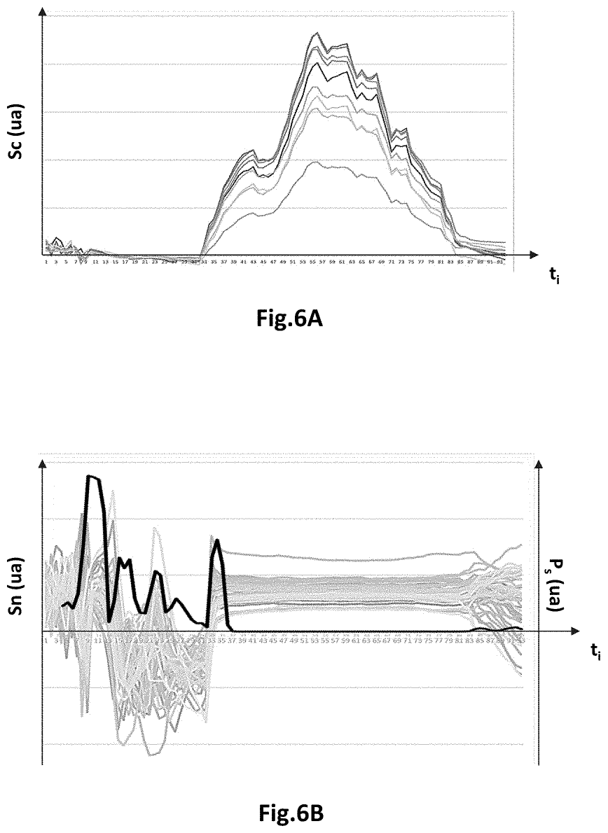

FIGS. 6A and 6B illustrate another example of the variation as a function of time (FIG. 6A) in the corrected vector Sc(tᵢ) computed from the useful vector Su(tᵢ), and one example of the variation as a function of time (FIG. 6B) in the normalized vector Sn(tᵢ) computed from the corrected vector Sc(tᵢ), and the variation as a function of time in the stability parameter Pₛₜ(tᵢ). These vectors were also obtained from degraded-profile sensorgrams. The corrected vector Sc(tᵢ) is computed here by applying conventional low-pass filtering to the useful vector Su(tᵢ). This approach allows the noise present in the useful vector Su(tᵢ) to be decreased. The stability parameter Pₛₜ(tᵢ) exhibits a first portion with large variations in the initial phase P1, followed by a transient portion with a high value at the start of the injecting phase P2, then a steady-state portion with an almost zero value in the injecting phase P2. This steady-state portion is here also used to characterize the target compounds. As above, it is thus possible to characterize the target compounds much more quickly than in the case of conventional characterizing methods, whether or not an equilibrium is reached between the absorption and desorption rates.

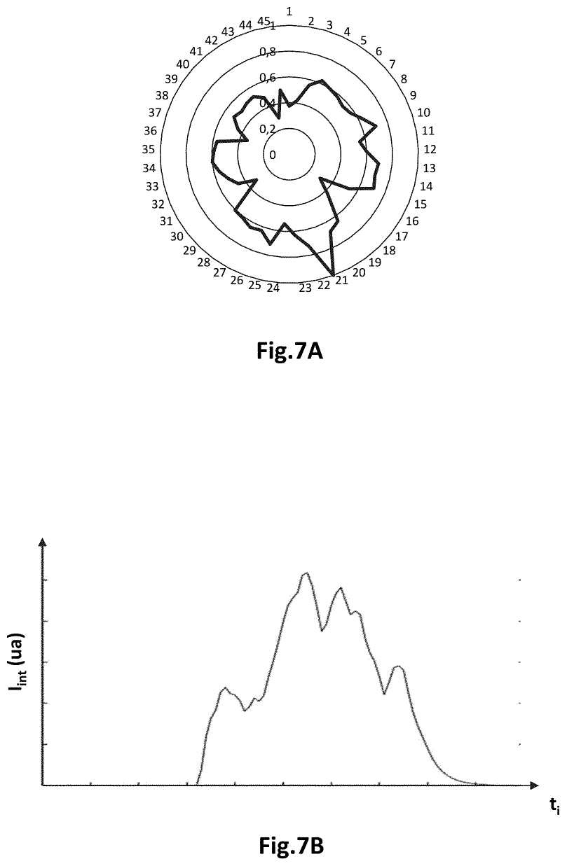

FIGS. 7A and 7B illustrate examples of information generated in the step of characterizing the target compounds. An interaction pattern may thus be generated (FIG. 7A), here one taking the form of a radar chart indicating the coordinate Snₖ(tᵢ) for each sensitive site of rank k ranging from 1 to 45, at the final measurement time tᵢ. In addition, the variation as a function of time in the interaction intensity Iᵢₙₜ(tᵢ) may also be determined (FIG. 7B). It corresponds here to the computation of the Euclidean norm of the normalized vector Sn(tᵢ) at the current time tᵢ. It thus provides the user with information on the instantaneous population of the target compounds among the receptors over time, this potentially being particularly important in certain fields, such as that of health. It will be noted that the integral of the interaction intensity Iᵢₙₜ(tᵢ) over the period of the injecting phase P2 allows an exposure level of the receptors to the target compounds to be deduced.

Additional information may also be deduced from the normalized vector Sn(tᵢ). Thus, some coordinates Snₖ(tᵢ) may exhibit a steady gradient (substantially constant slope), in the injecting phase P2. The value of the slope may also participate in the characterization of the target compounds. These parameters may be determined in a given period of the injecting phase P2.

Particular embodiments have just been described. Various modifications and variants will appear obvious to anyone skilled in the art.

As mentioned previously, the fluid sample containing the target compounds may be gaseous or liquid. The characterizing method is preferably implemented with gas samples by an electronic nose based on SPR imaging, but other analysis technologies may be implemented, such as analysis with electromechanical NEMS or MEMS resonators.

1. A method for characterizing target compounds, with an analyzing system comprising a measurement chamber intended to receive target compounds contained in a fluid sample, in which measurement chamber are located a plurality of distinct sensitive sites each comprising receptors that are able to interact with the target compounds, the method comprising the following steps:
fluidically supplying a fluid sample to the measurement chamber, this comprising an injecting phase P₂ in which the fluid sample is formed from a carrier fluid and the target compounds;
determining, in the supplying step, at a measurement time tᵢ, for each sensitive site, a measurement signal Sₖ(tᵢ) representative of the interactions between the target compounds and the receptors, k being the rank of the sensitive site in question, so as to obtain a measurement vector S(tᵢ) formed from the measurement signals Sₖ(tᵢ) acquired at the measurement time tᵢ;
computing, at the measurement time tᵢ, a normalized vector Sn(tᵢ) from the measurement vector S(tᵢ) at the measurement time tᵢ, and from a norm ∥S(tᵢ)∥ computed from the measurement vector S(tᵢ) at the measurement time tᵢ; and
reiterating the steps of determining measurement signals and of computing the normalized vector, while incrementing the measurement time, until a stability criterion is met, so as to obtain a characterization of the target compounds on the basis of the normalized vector Sn(tᵢ) at the measurement time tᵢ.

2. The method as claimed in claim 1, wherein:
the fluid-supplying step comprises, prior to the injecting phase P2, an initial phase P₁ in which the fluid sample is formed from the carrier fluid without the target compounds;
the step of determining the measurement signal Sₖ(tᵢ) comprises computing a useful vector Su(tᵢ), at the measurement time tᵢ, by subtracting from the measurement vector S(tᵢ) a reference vector S(Δt_{ref}) determined in the initial phase P1 in a predetermined measurement period Δt_{ref}; and
the normalized vector Sn(tᵢ) being computed from the useful vector Su(tᵢ).

3. The method as claimed in claim 1, wherein the step of determining the measurement signal Sₖ(tᵢ) comprises computing a corrected vector Sc(tᵢ) from the measurement vector S(tᵢ) with application of a low-pass filter or from a sum of the values of the measurement vector S(tᵢ) at the previous measurement times.

4. The method as claimed in claim 1, wherein the stability criterion comprises a comparison, at the measurement time tᵢ, of a stability parameter Pₛₜ(tᵢ) computed from the coordinates Snₖ(tᵢ) of the normalized vector Sn(tᵢ) in a moving window tᵢ-Tₛₜ, to a determined threshold value P_{st,th}.

5. The method as claimed in claim 4, wherein the stability parameter Pₛₜ(tᵢ) is the maximum among the variances computed at the measurement time tᵢ for the coordinates Snₖ(tᵢ-Tₛₜ) of the normalized vector Sn in a moving window tᵢ-Tₛₜ.

6. The method as claimed in claim 1, wherein the stability criterion comprises a comparison, at the measurement time tᵢ, of an injection parameter Pᵢₙⱼ(tᵢ) computed from the coordinates Sₖ(tᵢ) of the measurement vector S(tᵢ) in a moving window tᵢ-Tᵢₙⱼ, to a determined threshold value P_{inj,th}.

7. The method as claimed in claim 6, wherein the injection parameter Pᵢₙⱼ(tᵢ) is the maximum among the variances computed at the measurement time tᵢ for the coordinates Sₖ(tᵢ-Tᵢₙⱼ) of the measurement vector S in a moving window tᵢ-Tᵢₙⱼ.

8. The method as claimed in claim 1, wherein the norm ∥S(tᵢ)∥ is the Euclidean norm.

9. The method as claimed in claim 1, wherein the characterizing step comprises providing at least one parameter characteristic of a variation as a function of time in the Euclidean norm of the normalized vector Sn in the injecting phase P2.

10. The method as claimed in claim 9, wherein the characterizing step comprises computing an integral, over the duration of the injecting phase P2, of the Euclidean norm of the normalized vector Sn.

11. The method as claimed in claim 1, wherein the analyzing system is an electronic nose based on surface-plasmon-resonance imaging, or is an analyzing system comprising a plurality of distinct electromechanical resonators each forming one sensitive site.
